(19) United States
(12) Patent Application Publication
(10) Pub. No.: US 2007/0265335 A1
(43) Pub. Date: Nov. 15, 2007

(54) POLYMORPHS OF 5-CYCLOPROPYL-2-(4-FLUOROPHENYL)-6-[(2-HYDROXYETHYL)(METHYLSUFONYL)AMINO]-N-METHYL-1-BENZOFURAN-3-CARBOXAMIDE AND METHODS OF MAKING THE SAME

(75) Inventors: WEIGUO LIU, PARSIPPANY, NJ (US); QIUXIA WANG, NANUET, NY (US); WEI TONG, SUFFERN, NY (US); PANOLIL RAVEENDRANATH, MONROE, NY (US); SILVIO IERA, MONTREAL (CA)

Correspondence Address: FITZPATRICK CELLA HARPER & SCINTO, 30 ROCKEFELLER PLAZA, NEW YORK, NY, 10112, US

(73) Assignees: WYETH, Madison, NJ (US); VIROPHARMA INCORPORATED, Exton, PA (US)

(21) Appl. No.: 11/558,382

(22) Filed: Nov. 9, 2006

Related U.S. Application Data
(60) Provisional application No. 60/735,190, filed on Nov. 10, 2005.

(51) Int. Cl. A61K 31/343; A61P 31/14; C07D 307/78

(52) U.S. Cl. 514/469; 549/467

(57) ABSTRACT

Substantially pure polymorphic forms of 5-cyclopropyl-2-(4-fluorophenyl)-6-[(2-hydroxyethyl)(methylsulfonyl)amino]-N-methyl-1-benzofuran-3-carboxamide are produced by recrystallization in an organic solvent.

This application claims the benefit of U.S. Provisional Patent Application No. 60/735,190, filed Nov. 10, 2005, the entire disclosure of which is incorporated by reference herein.

### BACKGROUND OF THE INVENTION

1. Field of the Invention

This invention relates to various crystalline forms or polymorphs of 5-cyclopropyl-2-(4-fluorophenyl)-6-[(2-hydroxyethyl)(methylsulfonyl)amino]-N-methyl-1-benzofuran-3-carboxamide, as well as to methods of making the same, pharmaceutical compositions containing the same and methods of treatment using the same.

2. Related Background Art

Polymorphism, the ability of a molecule to crystallize into more than one crystal arrangement, can have a profound effect on the shelf life, solubility, formulation properties, and processing properties of a drug. In addition, the action of a drug can be affected by the polymorphism of the drug molecule. Different polymorphs can have different rates of uptake in the body, leading to lower or higher biological activity than desired. In extreme cases, an undesired polymorph can even be toxic. The occurrence of an unknown polymorphic form during manufacture can have an enormous impact.

Understanding and controlling polymorphism, then, gives a decided advantage in bringing new drugs to the marketplace. First and foremost, predicting any possible polymorphs for a drug product can be used to diminish the possibility of contamination during a drug's manufacture or storage by other polymorphic forms. Failure to catch contamination can have life-threatening consequences in some cases. Crystallizing an unintended polymorph during manufacture can mean weeks or even months of production downtime while scientists find and correct the cause of the new crystal form or go through another round of testing to obtain approval for the new form.

Second, understanding which crystal structures are possible allows researchers to maximize, in some cases, a compound's desired properties such as solubility, formulation properties, processing properties, and shelf life. Understanding these factors early in the development of a new drug may mean a more active, more stable, or more cheaply manufactured drug. The present invention relates to novel polymorphic forms of 5-cyclopropyl-2-(4-fluorophenyl)-6-[(2-hydroxyethyl)(methylsulfonyl)amino]-N-methyl-1-benzofuran-3-carboxamide, a known hepatitis C viral inhibitor, which provides numerous advantages.

### SUMMARY OF THE INVENTION

The present invention is directed to a substantially pure crystalline form A of 5-cyclopropyl-2-(4-fluorophenyl)-6-[(2-hydroxyethyl)(methylsulfonyl)amino]-N-methyl-1-benzofuran-3-carboxamide. Most preferably, this substantially pure crystalline form is characterized by the x-ray powder diffraction pattern of FIG. 1.

The present invention is further directed to a substantially pure crystalline form B of 5-cyclopropyl-2-(4-fluorophenyl)-6-[(2-hydroxyethyl)(methylsulfonyl)amino]-N-methyl-1-benzofuran-3-carboxamide. Most preferably, this substantially pure crystalline form is characterized by the x-ray powder diffraction pattern of FIG. 2.

The invention is still further directed to a method of producing a crystalline form of 5-cyclopropyl-2-(4-fluorophenyl)-6-[(2-hydroxyethyl)(methylsulfonyl)amino]-N-methyl-1-benzofuran-3-carboxamide comprising the steps of (a) providing a solution of 5-cyclopropyl-2-(4-fluorophenyl)-6-[(2-hydroxyethyl)(methylsulfonyl)amino]-N-methyl-1-benzofuran-3-carboxamide in at least one solvent; and (b) seeding the solution with 5-cyclopropyl-2-(4-fluorophenyl)-6-[(2-hydroxyethyl)(methylsulfonyl)amino]-N-methyl-1-benzofuran-3-carboxamide of the desired crystalline form. Other steps may include (c) cooling the solution and (d) adding an anti-solvent.

The present invention is further directed to a substantially pure crystalline form of 5-cyclopropyl-2-(4-fluorophenyl)-6-[(2-hydroxyethyl)(methylsulfonyl)amino]-N-methyl-1-benzofuran-3-carboxamide produced according to the above-noted inventive method.

The invention is still further directed to a pharmaceutical composition comprising (a) a therapeutically effective amount of a substantially pure crystalline form of 5-cyclopropyl-2-(4-fluorophenyl)-6-[(2-hydroxyethyl) (methylsulfonyl)amino]-N-methyl-1-benzofuran-3-carboxamide; and (b) at least one pharmaceutically acceptable carrier, diluent, vehicle or excipient.

The present invention is also directed to a method of treating hepatitis C comprising the step of administering to a subject in need of such treatment a therapeutically effective amount of a substantially pure crystalline form of 5-cyclopropyl-2-(4-fluorophenyl)-6-[(2-hydroxyethyl)(methylsulfonyl)amino]-N-methyl-1-benzofuran-3-carboxamide.

### BRIEF DESCRIPTION OF THE DRAWINGS

FIG. 1 shows the x-ray powder diffraction pattern for the polymorphic form A of 5-cyclopropyl-2-(4-fluorophenyl)-6-[(2-hydroxyethyl)(methylsulfonyl)amino]-N-methyl-1-benzofuran-3-carboxamide according to the present invention.

FIG. 2 shows the x-ray powder diffraction pattern for the polymorphic form B of 5-cyclopropyl-2-(4-fluorophenyl)-6-[(2-hydroxyethyl)(methylsulfonyl)amino]-N-methyl-1-benzofuran-3-carboxamide according to the present invention.

### DETAILED DESCRIPTION OF THE INVENTION

The hepatitis C virus inhibitor 5-cyclopropyl-2-(4-fluorophenyl)-6-[(2-hydroxyethyl)(methylsulfonyl)amino]-N-methyl-1-benzofuran-3-carboxamide (5-cyclopropyl-2-(4-fluorophenyl)-6-[(2-hydroxyethyl)(methylsulfonyl)amino]-N-methyl-1-benzofuran-3-carboxamide) is a potent inhibitor of the hepatitis C virus as disclosed in U.S. Patent Application Publication No. 2004-0162318. The structure of 5-cyclopropyl-2-(4-fluorophenyl)-6-[(2-hydroxyethyl) (methylsulfonyl)amino]-N-methyl-1-benzofuran-3-carboxamide is as follows:

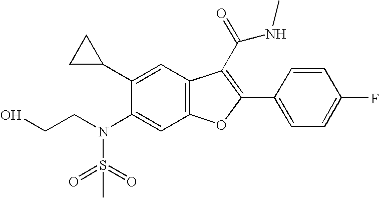

As noted above, knowledge of the potential polymorphic forms of 5-cyclopropyl-2-(4-fluorophenyl)-6-[(2-hydroxyethyl)(methylsulfonyl)amino]-N-methyl-1-benzofuran-3-carboxamide is useful in the development of a suitable dosage form because the failure to utilize a single polymorphic form during clinical or stability studies may result in the exact dosage form being used or studied not being comparable from one lot to another. Once chosen, it is important that a polymorphic form can be reproducibly prepared and remain unchanged for prolonged time periods in the dosage form developed. It is also desirable to have a process for producing 5-cyclopropyl-2-(4-fluorophenyl)-6-[(2-hydroxyethyl)(methylsulfonyl)amino]-N-methyl-1-benzofuran-3-carboxamide in high purity since the presence of impurities may produce undesired toxicological effects.

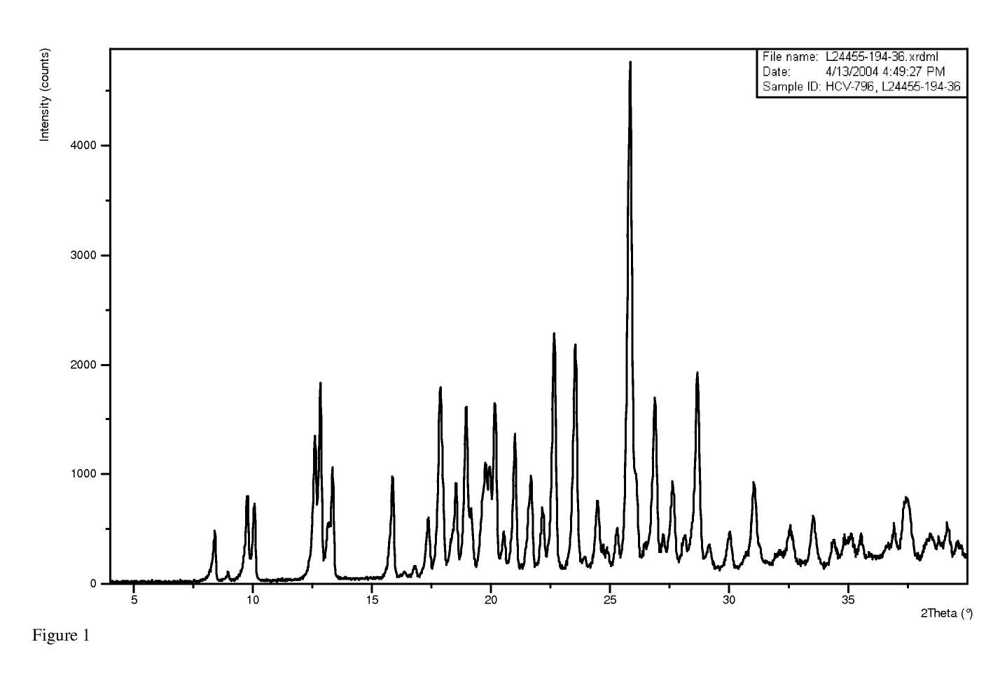

The first embodiment of the present invention is directed to a substantially pure polymorphic form A of 5-cyclopropyl-2-(4-fluorophenyl)-6-[(2-hydroxyethyl)(methylsulfonyl)amino]-N-methyl-1-benzofuran-3-carboxamide. The terms “polymorphic form”, “crystal modification”, “polymorph”, and “crystalline form” are used interchangeably herein. As used herein, the terms “isolated” and/or “substantially pure” mean more than 50% of the crystalline 5-cyclopropyl-2-(4-fluorophenyl)-6-[(2-hydroxyethyl)(methylsulfonyl)amino]-N-methyl-1-benzofuran-3-carboxamide or salt thereof is present in the form described herein and preferably at least 70%, more preferably at least 80%, and most preferably at least 90% of the crystalline form described herein is present. Preferably, the x-ray powder diffraction (XRPD) pattern for form A of 5-cyclopropyl-2-(4-fluorophenyl)-6-[(2-hydroxyethyl)(methylsulfonyl)amino]-N-methyl-1-benzofuran-3-carboxamide shows at least the following maxima: about 8.4°, 9.8°, 12.9°, 13.4° and 15.9° (2θ degrees). A particularly preferred embodiment of the present invention is directed to a substantially pure polymorphic form A as characterized by the XRPD pattern of FIG. 1. The peaks shown therein include: 8.4°, 9.0°, 9.8°, 10.1°, 12.6°, 12.9°, 13.2°, 13.4°, 15.9°, 16.4°, 16.8°, 17.4°, 17.9°, 18.4°, 18.5°, 19.0°, 19.2°, 19.7°, 19.8°, 20.0°, 20.2°, 20.5°, 21.0°, 21.7°, 22.2°, 22.6°, 23.6°, 24.0°, 24.5°, 24.7°, 24.9°, 25.3°, 25.9°, 26.0°, 26.5°, 26.9°, 27.3°, 27.6°, 28.1°, 28.7°, 27.2°, 30.0°, 30.7°, 31.0°, 31.9°, 32.1°, 32.5°, 33.6°, 34.4°, 34.8°, 35.1°, 35.6°, 36.7°, 36.9°, 37.5°, 38.4°, 38.8°, 39.2° and 39.6° (2θ degrees); one of ordinary skill in the art will readily understand that an about 0.1° to about 0.2° variation for peak positions is typical. The inventive polymorph appears to be a thermodynamically favored stable form as indicated by its high melting point of about 175° C. The inventive polymorph is non-hygroscopic when exposed to air for a period of about 1 week. This polymorph tends to settle quickly from suspension.

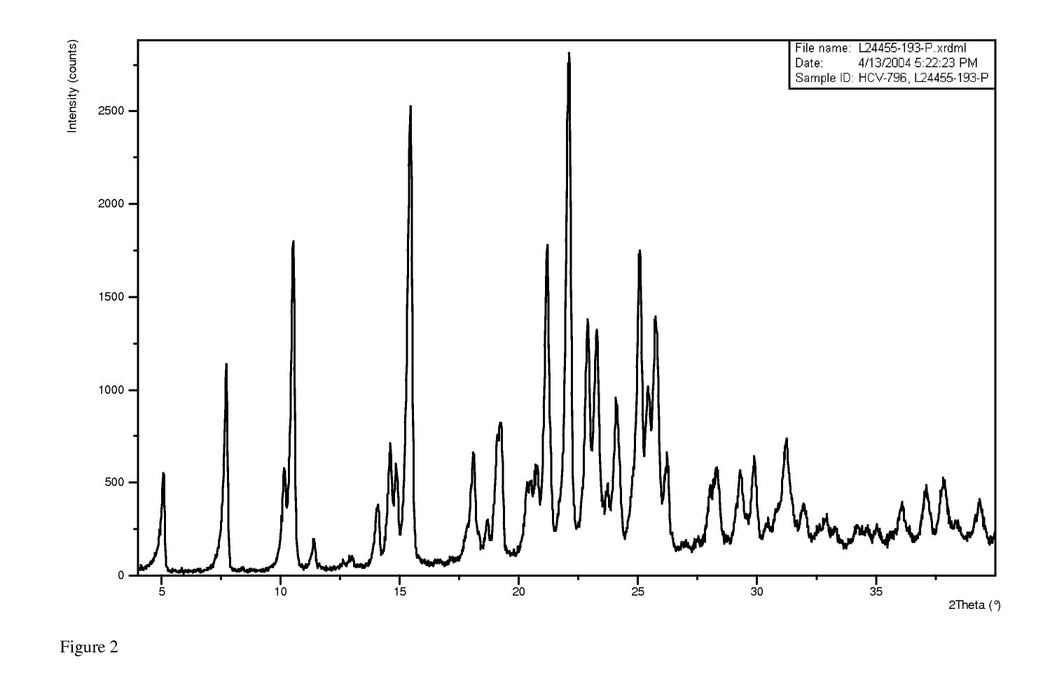

The second embodiment of the present invention is directed to a substantially pure polymorphic form B of 5-cyclopropyl-2-(4-fluorophenyl)-6-[(2-hydroxyethyl)(methylsulfonyl)amino]-N-methyl-1-benzofuran-3-carboxamide. Preferably, the XRPD pattern for form B of 5-cyclopropyl-2-(4-fluorophenyl)-6-[(2-hydroxyethyl)(methylsulfonyl)amino]-N-methyl-1-benzofuran-3-carboxamide shows at least the following maxima: about 5.1°, 7.7°, 10.6°, 14.1°, 14.6°, 14.9° and 15.5° (2θ degrees). A particularly preferred embodiment of the present invention is directed to a substantially pure polymorphic form B as characterized by the XRPD pattern of FIG. 2. The peaks shown therein include: 5.1°, 7.7°, 10.2°, 10.6°, 11.4°, 12.6°, 13.0°, 14.1°, 14.6°, 14.9°, 15.5°, 17.9°, 18.1°, 18.7°, 19.2°, 20.5°, 20.8°, 21.2°, 22.1°, 22.9°, 23.3°, 23.7°, 24.1°, 25.1°, 25.4°, 25.8°, 26.2°, 28.0°, 28.3°, 29.3°, 29.9°, 30.4°, 30.9°, 31.2°, 32.0°, 32.6°, 32.9°, 33.3°, 34.2°, 34.7°, 35.1°, 35.8°, 36.1°, 37.1°, 37.8°, 38.4° and 39.3° (2θ degrees); one of ordinary skill in the art will readily understand that an about 0.1° to about 0.2° variation for peak positions is typical. This inventive polymorph has a melting point of about 180° C. and tends not to settle quickly from suspension.

The third embodiment of the present invention is directed to a method of producing a substantially pure crystalline form of 5-cyclopropyl-2-(4-fluorophenyl)-6-[(2-hydroxyethyl)(methylsulfonyl)amino]-N-methyl-1-benzofuran-3-carboxamide by recrystallizing crude 5-cyclopropyl-2-(4-fluorophenyl)-6-[(2-hydroxyethyl)(methylsulfonyl)amino]-N-methyl-1-benzofuran-3-carboxamide using at least one solvent and 5-cyclopropyl-2-(4-fluorophenyl)-6-[(2-hydroxyethyl)(methylsulfonyl)amino]-N-methyl-1-benzofuran-3-carboxamide seed of the desired crystalline form. More particularly, the inventive method comprises the steps of (a) providing a solution of 5-cyclopropyl-2-(4-fluorophenyl)-6-[(2-hydroxyethyl)(methylsulfonyl)amino]-N-methyl-1-benzofuran-3-carboxamide in at least one solvent; and (b) seeding the solution with 5-cyclopropyl-2-(4-fluorophenyl)-6-[(2-hydroxyethyl)(methylsulfonyl)amino]-N-methyl-1-benzofuran-3-carboxamide of the desired crystalline form. This process is suitable for the production of substantially pure crystalline forms of 5-cyclopropyl-2-(4-fluorophenyl)-6-[(2-hydroxyethyl)(methylsulfonyl)amino]-N-methyl-1-benzofuran-3-carboxamide in high purity in large scale. The polymorphic form A of 5-cyclopropyl-2-(4-fluorophenyl)-6-[(2-hydroxyethyl)(methylsulfonyl)amino]-N-methyl-1-benzofuran-3-carboxamide prepared according to the method of the third embodiment shows a higher chemical purity (>98%) than other known forms.

In step (a) of the inventive method, a solution of 5-cyclopropyl-2-(4-fluorophenyl)-6-[(2-hydroxyethyl)(methylsulfonyl)amino]-N-methyl-1-benzofuran-3-carboxamide in at least one solvent is provided. It is important to note that this solution can be provided by utilizing 5-cyclopropyl-2-(4-fluorophenyl)-6-[(2-hydroxyethyl)(methylsulfonyl)amino]-N-methyl-1-benzofuran-3-carboxamide in situ, e.g., by using a reaction mixture from the actual synthesis of 5-cyclopropyl-2-(4-fluorophenyl)-6-[(2-hydroxyethyl)(methylsulfonyl)amino]-N-methyl-1-benzofuran-3-carboxamide, or that this solution can be provided by dissolving previously isolated crude 5-cyclopropyl-2-(4-fluorophenyl)-6-[(2-hydroxyethyl)(methylsulfonyl)amino]-N-methyl-1-benzofuran-3-carboxamide or 5-cyclopropyl-2-(4-fluorophenyl)-6-[(2-hydroxyethyl)(methylsulfonyl)amino]-N-methyl-1-benzofuran-3-carboxamide of any crystalline, salt or other form in at least one solvent.

Step (a) is typically and preferably conducted at an elevated temperature in order to form a solution. The elevated temperature of step (a) is typically about 5° C. below the boiling point of the solvent(s) used. One of ordinary skill in the art will readily understand that step (a) could also be conducted using greater amounts of solvent instead of an elevated temperature (or some combination of these two parameters); however, given the impracticality of using large amounts of solvent on a large scale, it is preferred to conduct step (a) at an elevated temperature to achieve a solution.

Solvents suitable for use in the present invention include, without limitation, isopropyl alcohol, ethanol, ethyl acetate, acetonitrile, acetone, tetrahydrofuran, toluene, water, and combinations thereof. In certain preferred embodiments of the invention, ethanol alone, ethyl acetate alone, or a combination of ethanol and ethyl acetate is used. Typically, the at least one solvent is employed in step (a) in an amount ranging from about 3 to about 20 times, more preferably from about 5 to about 10 times, by weight of the amount of 5-cyclopropyl-2-(4-fluorophenyl)-6-[(2-hydroxyethyl)(methylsulfonyl)amino]-N-methyl-1-benzofuran-3-carboxamide.

In step (b) of the inventive method, the solution is seeded with 5-cyclopropyl-2-(4-fluorophenyl)-6-[(2-hydroxyethyl)(methylsulfonyl)amino]-N-methyl-1-benzofuran-3-carboxamide of the desired crystalline form. More particularly, if form A of 5-cyclopropyl-2-(4-fluorophenyl)-6-[(2-hydroxyethyl)(methylsulfonyl)amino]-N-methyl-1-benzofuran-3-carboxamide is desired, then seeding in this step is carried out with form A 5-cyclopropyl-2-(4-fluorophenyl)-6-[(2-hydroxyethyl)(methylsulfonyl)amino]-N-methyl-1-benzofuran-3-carboxamide seed crystals; likewise, if form B of 5-cyclopropyl-2-(4-fluorophenyl)-6-[(2-hydroxyethyl)(methylsulfonyl)amino]-N-methyl-1-benzofuran-3-carboxamide is desired, then seeding in this step is carried out with form B 5-cyclopropyl-2-(4-fluorophenyl)-6-[(2-hydroxyethyl)(methylsulfonyl)amino]-N-methyl-1-benzofuran-3-carboxamide seed crystals.

In a particularly preferred embodiment of the present invention, step (b) is conducted in conjunction with a step (c) cooling the solution. It is important to note that, when step (c) is employed, step (c) may precede or follow step (b) or may be conducted simultaneously with step (b) in order to crystallize the desired crystalline form of 5-cyclopropyl-2-(4-fluorophenyl)-6-[(2-hydroxyethyl)(methylsulfonyl)amino]-N-methyl-1-benzofuran-3-carboxamide.

Typically, an amount of seed ranging from about 0.01% to about 2%, more typically ranging from about 0.1% to about 1%, by weight of the 5-cyclopropyl-2-(4-fluorophenyl)-6-[(2-hydroxyethyl)(methylsulfonyl)amino]-N-methyl-1-benzofuran-3-carboxamide (in solution in step (a)) is employed. In a particularly preferred embodiment of this invention, step (b) is carried out repeatedly, i.e., seeding is accomplished in portions. Furthermore, step (c) may be carried out repeatedly or continuously in conjunction with step (b), i.e., cooling may be continuous as various portions of seed are added or the solution may be cooled then seeded (or vice versa) and then cooled further and seeded again (or vice versa), etc.

Typically, the solution is cooled until crystallization is achieved. In preferred embodiments of the invention, the solution is cooled to about room temperature or cooled to about 0° C. During step (b) (and step (c) when present), the solution is preferably agitated using any suitable means; in fact, it is preferable to stir throughout the entire process, but amount and intensity of stirring can be determined by one of skill in the art. Recrystallization of form A of 5-cyclopropyl-2-(4-fluorophenyl)-6-[(2-hydroxyethyl)(methylsulfonyl)amino]-N-methyl-1-benzofuran-3-carboxamide is facilitated by prolonged agitation, while recrystallization of form B is facilitated by quick cooling.

In another preferred embodiment of this invention, crystallization of the desired crystalline form is further facilitated by an optional step (d) adding an anti-solvent. An anti-solvent preferred for use in step (d) is heptane, though one of ordinary skill in this art will readily appreciate that other suitable anti-solvents could be identified and used, i.e., hexane, keeping in mind certain regulatory concerns. Step (d) is conducted in conjunction with step (b), and more preferably in conjunction with step (c) as well. In other words, any combination of seeding, cooling and adding anti-solvent are contemplated for use in the inventive method. These steps can be carried out repeatedly, in succession in any order, simultaneously, etc.

The present inventive method may also include optional steps which serve to isolate the substantially pure crystalline form of 5-cyclopropyl-2-(4-fluorophenyl)-6-[(2-hydroxyethyl)(methylsulfonyl)amino]-N-methyl-1-benzofuran-3-carboxamide. Such optional steps include filtration and/or drying of the substantially pure crystalline form of 5-cyclopropyl-2-(4-fluorophenyl)-6-[(2-hydroxyethyl)(methylsulfonyl)amino]-N-methyl-1-benzofuran-3-carboxamide. Filtration may be accomplished using any suitable means and drying is preferably accomplished overnight at 50° C., though higher temperatures can be employed so long as the temperature is below the melting point of the substantially pure crystalline form of 5-cyclopropyl-2-(4-fluorophenyl)-6-[(2-hydroxyethyl)(methylsulfonyl)amino]-N-methyl-1-benzofuran-3-carboxamide.

The fourth embodiment of the present invention is directed to a substantially pure crystalline form of 5-cyclopropyl-2-(4-fluorophenyl)-6-[(2-hydroxyethyl)(methylsulfonyl)amino]-N-methyl-1-benzofuran-3-carboxamide produced according to the method of the third embodiment.

The fifth embodiment of the present invention is directed to a pharmaceutical composition comprising (a) a therapeutically effective amount of a substantially pure crystalline form of 5-cyclopropyl-2-(4-fluorophenyl)-6-[(2-hydroxyethyl)(methylsulfonyl)amino]-N-methyl-1-benzofuran-3-carboxamide; and (b) at least one pharmaceutically acceptable carrier, diluent, vehicle or excipient. The substantially pure crystalline form of 5-cyclopropyl-2-(4-fluorophenyl)-6-[(2-hydroxyethyl)(methylsulfonyl)amino]-N-methyl-1-benzofuran-3-carboxamide is preferably form A or form B (i.e., the substantially pure crystalline form of any of the first, second and fourth embodiments described above) and is most preferably characterized by the XRPD pattern of FIG. 1 or 2.

A “therapeutically effective amount” is intended to mean the amount of the inventive polymorph that, when administered to a subject in need thereof, is sufficient to effect treatment for disease conditions alleviated by the inhibition of hepatitis C virus. The amount of a given compound of the invention that will be therapeutically effective will vary depending upon factors such as the disease condition and the severity thereof, the identity of the subject in need thereof, etc., which amount may be routinely determined by artisans of ordinary skill in the art.

The at least one pharmaceutically acceptable carrier, diluent, vehicle or excipient can readily be selected by one of ordinary skill in the art and will be determined by the desired mode of administration. Illustrative examples of suitable modes of administration include oral, nasal, parenteral, topical, transdermal, and rectal. The pharmaceutical compositions of this invention may take any pharmaceutical form recognizable to the skilled artisan as being suitable. Suitable pharmaceutical forms include solid, semisolid, liquid, or lyophilized formulations, such as tablets, powders, capsules, suppositories, suspensions, liposomes, and aerosols.

The sixth embodiment of the present invention is directed to a method of treating hepatitis C comprising the step of administering to a subject in need of such treatment a therapeutically effective amount of a substantially pure crystalline form of 5-cyclopropyl-2-(4-fluorophenyl)-6-[(2-hydroxyethyl)(methylsulfonyl)amino]-N-methyl-1-benzofuran-3-carboxamide. The substantially pure crystalline form of 5-cyclopropyl-2-(4-fluorophenyl)-6-[(2-hydroxyethyl)(methylsulfonyl)amino]-N-methyl-1-benzofuran-3-carboxamide is preferably form A or form B (i.e., the substantially pure crystalline form of any of the first, second and fourth embodiments described above) and is most preferably characterized by the XRPD pattern of FIG. 1 or 2. As noted above, illustrative modes of administration include oral, nasal, parenteral, topical, transdermal, and rectal. Administration of the stable crystalline form may be accomplished by administration of a pharmaceutical composition of the fourth embodiment of the invention or via any other effective means.

Specific embodiments of the invention will now be demonstrated by reference to the following examples. It should be understood that these examples are disclosed solely by way of illustrating the invention and should not be taken in any way to limit the scope of the present invention.

### EXAMPLE 1

5-Cyclopropyl-2-(4-fluorophenyl)-6-[(2-hydroxyethyl)(methylsulfonyl)amino]-N-methyl-1-benzofuran-3-carboxamide (30.1 g) was charged into 207 g isopropyl alcohol, and the mixture was dissolved by heating to 70° C. The solution was cooled to ˜40-50° C. and then passed through a filter to remove potential mechanic impurities such as pieces of filter paper, dust, etc. The filtrate was heated to 70-80° C. for 10 min and then held at 50° C. for 2-3 hours before being air cooled to room temperature. After over weekend agitation at room temperature, a solid (form A of 5-cyclopropyl-2-(4-fluorophenyl)-6-[(2-hydroxyethyl)(methylsulfonyl)amino]-N-methyl-1-benzofuran-3-carboxamide) was filtered and dried under nitrogen flow at room temperature. Further drying in an oven at 50-90° C. under vacuum did not reduce the amount of the residual solvent (isopropyl alcohol, 0.9%). Recovery yield: 27.1 g, 90%. An x-ray powder diffraction pattern (FIG. 1) was obtained.

### EXAMPLE 2

A polymorphic form of 5-cyclopropyl-2-(4-fluorophenyl)-6-[(2-hydroxyethyl)(methylsulfonyl)amino]-N-methyl-1-benzofuran-3-carboxamide was produced in various solvent systems as set forth in Table 1 below.

TABLE 1

| solvent | crystallization | DSC | | TGA | microscopy |
|---|---|---|---|---|---|
| | | T_{onset 1} (° C.) | T_{onset 2} (° C.) | weight loss | |
| Isopropyl | Cooling | — | 175.7 | — | Crystalline |
| alcohol | | | | | |
| Isopropyl | Cooling | — | 176.6 | — | Thin needles, |
| alcohol | | | | | crystalline (dry) |
| Ethanol | Cooling | 57.1 | 143.2 | 1.0% before | Highly crystalline |
| | | | | 110° C. | |
| Ethyl acetate | Cooling | 39.1 | 112.2 | — | Aggregates, highly |
| | | | | | crystalline |
| Acetonitrile | Cooling | 92.3* | — | — | Thin rod crystals, |
| | | | | | highly crystalline |
| Ethanol | Anti by water | 53.4 | 140.1 | — | Thin needles, highly |
| | | | | | crystalline |
| Acetone | Anti by water | 68.6 | 112.6 | — | Thin rod crystals, |
| | | | | | highly crystalline |
| Acetone | Anti by heptane | 31.8 | 107.4 | 1.0% before | Tiny particles, |
| | | | | 60° C., 5.2% | highly crystalline |
| | | | | 60-100° C. | |
| Acetonitrile | Anti by heptane | 95.2 | 113.5 | 4.9% before | Tiny thin needle |
| | | | | 93° C. | crystals, crystalline |
| Acetonitrile | Anti by water | 69.9 | 111.7 | 4.6% before | Needle crystals, |
| | | | | 100° C. | highly crystalline |
| Isopropyl | Anti by water | 71.2 | 108.3 | — | Tiny needle crystals, |
| alcohol | | | | | crystalline |
| Isopropyl | Evaporation | — | 177.6 | — | Not highly crystalline |
| alcohol | | | | | |
| Acetone | Evaporation | 89.3 | 176.1 | — | Plate like, not |
| | | | | | crystalline |
| Ethanol | Evaporation | 138.9 | — | 0.5% bf | Partially |
| | | | | 62° C., 1.4% | crystalline |
| | | | | 62-122° C., 1.4% | |
| | | | | 122-202° C. | |
| THF | Evaporation | 76.3 | 174.6 | — | Not crystalline |
| Toluene | Slurry | 128.9 | — | 3.4% before | Long needle crystals, |
| | | | | 139° C., 1.8% | highly crystalline |
| | | | | 139-225° C. | |
| Water | Slurry | 37.1 | 144.6 | — | Needle crystals, |
| | | | | | highly crystalline |
| tert-butyl | Slurry | — | 178.0 | — | Long needle crystals, |
| methyl ether | | | | | highly crystalline |
| Ethanol | Slurry | 50.2 | 146.4 | 1.5% before | Tiny crystals, |
| | | | | 136° C. | highly crystalline |
| Ethyl acetate | Slurry | 28.5 | 116.6 | — | Non needle, |
| | | | | | highly crystalline |
| Ethyl acetate + | Slurry | 27.7 | 119.7 | — | Non needle, |
| water | | | | | highly crystalline |
| acetonitrile + | Slurry | 80.9 | 111.2 | — | Tiny crystals, |
| water | | | | | highly crystalline |
| Acetone + | Slurry | 73.8 | 113.5 | 3.6% before | Needle crystals, |
| water | | | | 81° C. | highly crystalline |
| Acetone + | Slurry | 28.3 | 106.9 | 1.4% bf | Aggregates and non |
| heptane | | | | 60° C., 4.6% | needle, crystalline |
| | | | | 60-117° C., 0.5% | |
| | | | | 134-192° C. | |
| Isopropyl | Slurry | 150.9 | 165.8 | — | Needle crystals, |
| alcohol at 5° C. | | | | | highly crystalline |
| Isopropyl | Slurry | 146.5 | 174.9 | — | Needle crystals, |
| alcohol at 25° C. | | | | | highly crystalline |
| Isopropyl | Slurry | — | 176.7 | — | Long needle, |
| alcohol at 45° C. | | | | | highly crystalline |
| Isopropyl | Slurry | — | 177.8 | — | Long needle crystals, |
| alcohol at 65° C. | | | | | highly crystalline |

### EXAMPLE 3

Prepare slurry of celite (0.02 kg) and ethyl acetate (0.09 kg, 0.10 L). Filter the celite slurry through filter media in a tared Büchner funnel, mounted on a tared 4-L suction flask. Discard the filtrates.

| | |
|---|---|
| Filter inside diameter: | (10 cm). |
| Celite cake thickness: | (2 cm). |

Clarify the contents of the 6-L reactor containing 5-cyclopropyl-2-(4-fluorophenyl)-6-[(2-hydroxyethyl)(methylsulfonyl)amino]-N-methyl-1-benzofuran-3-carboxamide through the pre-coated filter into the suction flask using vacuum. Maintain suction until the filtration essentially stops.

| | |
|---|---|
| Filtration time: | (5 min) |

Filtrate is yellowish, clear

Rinse the reactor, then the filter cake with ethyl acetate (2×0.90 kg, 2×0.10 L) at 65 to 70° C. Allow the rinse to percolate through the filter cake for 10 min before applying vacuum.

| | | |
|---|---|---|
| Rinse filtration time: | (5 | min). |
| Combined filtrates volume: | (2.9 | L). |
| Combined filtrates mass: | (2.8 | kg). |
| Cake thickness: | (2 | cm). |

Transfer the filtrates from the suction flask to the 3-L reactor and rinse using solvent (0.05 kg, 0.05 L). Hold the wet cake for disposal at the end of the process. Equip the reactor with distillation head connected with condenser and 2-L receiving flask immersed into an ice-water bath. Pre-mark the receiving flask at 1.9-L level. Concentrate the solution from previous step via atmospheric distillation to a volume of ˜0.8-0.9 L by collecting ˜1.9 L distillate in the receiver.

| | |
|---|---|
| Batch temperature | (75 to 80° C.). |
| Jacket temperature | (85 to 95° C.). |
| Vapor temperature | (76 to 78° C.). |
| Distillation time | (2 h 15 min). |
| Agitation speed | (185-195 rpm). |
| Final reaction mixture volume | (0.9 L). |

Expect formation of clear solution. Charge 1.08 kg, 1.20 L of prefiltered alcohol 1J1 to the reactor over 20 min while maintaining the temperature at 70-80° C.

| Jacket temperature | (75 to 85° | C.). |
|---|---|---|
| Initial temperature | (75° | C.). |
| Final temperature | (78° | C.). |

Continue the distillation until another 0.39 kg, 0.42 L of distillate is collected to a volume of 1.6 L.

| | |
|---|---|
| Batch temperature | (72 to 78° C.). |
| Jacket temperature | (90 to 95° C.). |
| Vapor temperature | (74 to 80° C.). |
| Distillation time | (45 min). |
| Agitation speed | (190-200 rpm). |
| Final reaction mixture volume | (2.2 L). |

Charge 0.55 kg, 0.80 L of prefiltered heptane to the reactor over 20 min while maintaining the temperature at 70-80° C. Expect formation of clear solution. Cool the contents of the 6-L reactor to 65 to 70° C. while stirring at low speed at an approximate rate of 1° C./min. Seed the contents of the reactor at 5° C. intervals, starting at 70° C. using seeds (0.0002 kg for each seeding).

| | | |
|---|---|---|
| Agitation speed | (170-180 | rpm). |
| The solution seeded at | (70° | C.). |
| Total amount of seeds used | (0.0002 | kg). |
| The product starts to crystallize at | (60° | C.). |
| Time from the first seeding | (10 | min). |

Empty the receiver.

| | |
|---|---|
| Distillate volume | (2.4 L). |

Set the distillate aside for disposal. Cool the mixture to 45 to 50° C. Expect formation of a thin suspension. Stir the mixture at 45 to 50° C. for a minimum of 2 h at low speed.

| | |
|---|---|
| Agitation speed | (170-180 rpm). |

Expect formation of suspension. Cool the contents of the 3-L reactor to 20 to 25° C. over a minimum of 0.5 h at low stirring.

| | | |
|---|---|---|
| Initial temperature: | (48° | C.). |
| Final temperature: | (22° | C.). |
| Cooling duration: | (0.5 | h). |
| Jacket temperature | (5 to 10° | C.). |
| Agitation speed | (170-180 | rpm). |

Expect formation of suspension. Stir the mixture at 20 to 25° C. for a minimum of 16 h at low speed.

| | |
|---|---|
| Agitation speed | (170-180 rpm). |

Mixture can be held overnight with stirring at 20 to 25° C. Prepare a 15 cm diameter tared Büchner filter funnel with filter media connected with tared 4-L filter flask. Filter the mixture using house vacuum, and collecting the solids and filtrate. Filtrate can be re-circulated for transfer of solids.

| | | |
|---|---|---|
| Filtration time: | (5 | min). |
| Cake height: | (3 | cm). |
| Filtrate Volume: | (2.2 | L). |

Rinse the reactor with prefiltered alcohol 1J1:heptane (1:1) solution (2×0.08 kg, 2×0.10 L) at 20° C., and then use this rinse as a wash on the filter cake. Allow wash to percolate for a minimum of 10 min before applying full suction. Maintain suction until filtration essentially stops. Wash cake with prefiltered alcohol:heptane (1:1) solution (0.08 kg, 0.10 L). Hold filtrates/washes for waste treatment. Allow wash to percolate for a minimum of 10 min before applying full suction. Maintain suction until filtration essentially stops.

| | | |
|---|---|---|
| Total rinse/wash filtration time: | (15 | min). |
| Cake height after washes: | (3 | cm). |
| Total volume of washes: | (0.3 | L). |

Oven dry the product in a tared glass container in a vacuum oven with nitrogen bleed at 55-60° C. maintaining vacuum at 5-10 mm Hg for a minimum of 24 h. Terminate drying at loss on drying of less than 0.5%.

| | | |
|---|---|---|
| Cake wet weight: | (0.19 | kg). |
| Drying time | (24 | h). |
| Cake dry weight: | (0.183 | kg) - 82.5% yield |

### EXAMPLE 4

To a 1-L cylindrical reactor equipped with a circulating bath (set at 21-22° C. initially), a mechanical stirrer, a distillation head, a nitrogen inlet and a baffle, crude 5-cyclopropyl-2-(4-fluorophenyl)-6-[(2-hydroxyethyl)(methylsulfonyl)amino]-N-methyl-1-benzofuran-3-carboxamide (0.080 kg, 0.179 mol) is charged. Next ethanol 1H (0.454 kg, 0.574 L) is charged to the reactor. A thin suspension will likely result. Agitation (216 rpm) is started and then ethyl acetate (0.237 kg, 0.262 L) is charged to the reactor. The temperature will decrease (to about 16.7 C) and a thin suspension will likely result. The mixture is heated with stirring to 70 to 75° C. over a minimum of 25 minutes by setting the jacket temperature at 75° C. A hazy solution will likely result. The mixture is stirred at 70 to 75° C. for a minimum of 20 minutes. A hazy solution will likely result. The contents of the reactor are then cooled to 40 to 45° C. over a minimum of 30 minutes with agitation. The mixture will likely remain a hazy solution. The contents of the reactor are clarified through a pre-packed bed of Celite into a tared 2-L suction flask using vacuum. A filtrate sample is pulled to determine solvent ratio by NMR.

| Weight of celite: | 0.015 | kg |
|---|---|---|
| Diameter of Buchner/fritted filter: | 4.3 | cm |
| Celite cake thickness: | 2.7 | cm |
| Filtration time: | 2 | min |
| Description of filtrate (color/clarity, etc.): | yellow clear solution | |
| Weight of filtrate: | 0.763 | kg |

| | |
|---|---|
| Solvent ratio EtOac:EtOH: | 1:3.26 mol/mol, 0.587:1 wt/wt |

The reactor is then rinsed and next the cartridge/filter cake is rinsed with a mixture of solvents (ethanol 1-H 0.0453 kg and ethyl acetate 0.0237 kg) while maintaining the jacket temperature at 45° C. The rinse is allowed to percolate for ˜1 min before applying vacuum. A sample is pulled to determine solvent ratio.

| | | |
|---|---|---|
| Rinse filtration time: | <1 | min |
| Combined filtrates: | 0.804 | kg |

| | |
|---|---|
| Solvent ratio EtOAc:EtOH: | 1:3.33 mol/mol, 0.575:1 wt/wt |

The reactor is cleaned with 150 g ethanol 1H. The filtrates are transferred from the suction flask to the reactor. This mixture is heated with stirring by setting the jacket temperature ramping from 22° C. to 95 in 45 minutes. A sample is collected from the initial distillate to determine the solvent ratio.

| | | |
|---|---|---|
| Initial temperature: | 23.3° | C. |
| Batch temperature of first drop: | 74° | C. |
| Jacket temperature at first drop: | 83.6° | C. |
| Heating time to see first drop: | 38 | min |

| | |
|---|---|
| Solvent ratio in first drop: | 1:1.55 mol/mol, 1.24:1 wt/wt |
| EtOAc:EtOH: | |

A clear solution should result. When the jacket temperature reaches 95° C. (about another 7 minutes), the jacket temperature is maintained at 95° C. for 25 minutes and then reset to 100° C. to accelerate the distillation. The time for jacket temperature to rise from 95 to 100° C. is about 3 minutes. Distillation is continued at T_{jacket}=100° C. for 19 minutes. The total distillate=0.290 kg, V=340 ml, the remaining volume is 0.680 L. NMR samples are pulled from the distillate and batch to determine the solvent ratio.

| | | |
|---|---|---|
| Final batch temperature: | 75° | C. |
| Final vapor temperature: | 73.8° | C. |

| | |
|---|---|
| Distillate weight and volume: | 290 g, 340 ml |

The jacket temperature is then set to 70° C. The jacket temperature dropped from 100° C. to 70° C. in 5 minutes. The mixture is stirred for an additional 5 minutes, and NMR samples are pulled from the distillate and batch to determine the solvent ratio.

| | |
|---|---|
| Distillate solvent ratio EtOAc:EtOH: | 1:1.91 mol/mol, 1:1 wt/wt |
| Batch solvent ratio EtOAc:EtOH: | 1:5.27 mol/mol, 0.363:1 wt/wt |

Next, heptane (0.219 kg) is charged to the reactor.

| | | |
|---|---|---|
| Initial temperature: | 68.5° | C. |
| Final temperature: | 63° | C. |
| Lowest temperature: | 61° | C. |
| Jacket temperature: | 70° | C. ° C. |

A clear solution will likely result. 5-Cyclopropyl-2-(4-fluorophenyl)-6-[(2-hydroxyethyl)(methylsulfonyl)amino]-N-methyl-1-benzofuran-3-carboxamide seed (0.05 g) is charged to the reactor.

| | |
|---|---|
| Batch temperature: | 65° C. |
| Jacket temperature: | 70° C. |

The mixture will likely start turning turbid slowly. The mixture will become very cloudy in 25 minutes, and the batch temperature was 62. The jacket temperature is then set to ramp down from 70° C. to 0° C. in 3 h and maintain the jacket temperature at room temperature overnight. (In 25 minutes, when batch temperature is 62° C., the mixture became turbid.) The mixture is released from the reactor.

| | | |
|---|---|---|
| Batch temperature: | 0° | C. |
| Weight of slurry: | 0.718 | kg |

The mixture is filtered on a Büchner funnel using vacuum pump/nitrogen pressure. Filtrate cannot be circulated for transfer of solids.

| Filter media: | | |
|---|---|---|
| Cake diameter: | 9.5 | cm |
| Cake height: | 1.5 | cm |
| Wet cake weight: | 0.095 | kg |
| Filtration time: | 2.5 | min |
| Filtrate weight: | 0.605 | kg |

The filter cake is washed with ethanol 1H 0.080 kg. The wash is combined with mother liquor from previous step. The reactor was rinsed with this wash.

| | Wash # | | |
|---|---|---|---|
| | 1 | 2 | 3 |
| Solvent | EtOH 1H | EtOH 1H | |
| Amount of solvent (kg, L) | 0.080 kg | 0.080 kg | |
| Percolation time (min) | 10 min | 10 min | |
| Filtration time (min) | 2 min | 2 min | |

| | |
|---|---|
| Cake height after all washes (cm) | 1.5 cm |
| Wet cake weight after all washes (kg) | 0.095 kg |
| Combined filtrate weight (kg) | 0.760 kg |
| Precipitation/separation in mother liquor | No |

The wet cake is suction dried under nitrogen in the tared Büchner funnel using a vacuum pump for 2 h.

| | | |
|---|---|---|
| Cake wet weight: | 0.095 | kg |
| Drying time: | 2 | h |
| Cake dry weight: | 0.0728 | kg |

The product is oven dried in a suitable tared container in a vacuum oven with nitrogen bleed at 55° C. maintaining vacuum at 20 mbar for a minimum of 4 h until a loss on drying of less than 1% is obtained.

| | | |
|---|---|---|
| Initial weight: | 0.0728 | kg |
| Drying time: | 4 | h |
| Cake dry weight: | 0.0728 | kg (91% yield) |

### EXAMPLE 5

5-Cyclopropyl-2-(4-fluorophenyl)-6-[(2-hydroxyethyl)(methylsulfonyl)amino]-N-methyl-1-benzofuran-3-carboxamide (20 g) was added into a 250 mL flask containing 75.5 g ethanol and 39 g ethyl acetate. The mixture was heated to 70° C. A solution was formed when the temperature reached 63° C. Heptane (55 g) was then added to the flask at about 65° C. in about 10 min followed by 11 mg of stable polymorph form A seed. The seed stayed as solid at 65° C. after 30 minutes stirring. The mixture was then cooled to room temperature. The slurry settled very slowly when agitation was stopped; therefore it was re-heated to 65° C. and stirred for 35 minutes. Upon cooling to room temperature again, the slurry settled on the bottom very quickly. Upon filtration, 16.5 g product of stable polymorph form A was obtained.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A substantially pure crystalline form A of 5-cyclopropyl-2-(4-fluorophenyl)-6-[(2-hydroxyethyl)(methylsulfonyl)amino]-N-methyl-1-benzofuran-3-carboxamide.

2. The substantially pure crystalline form of claim 1, wherein the substantially pure crystalline form is characterized by an x-ray powder diffraction pattern having at least the following maxima: about 8.4°, 9.8°, 12.9°, 13.4° and 15.9° (2θ degrees).

3. The substantially pure crystalline form of claim 1, wherein the substantially pure crystalline form is characterized by the x-ray powder diffraction pattern of FIG. 1.

4. A substantially pure crystalline form B of 5-cyclopropyl-2-(4-fluorophenyl)-6-[(2-hydroxyethyl)(methylsulfonyl)amino]-N-methyl-1-benzofuran-3-carboxamide.

5. The substantially pure crystalline form of claim 4, wherein the substantially pure crystalline form is characterized by an x-ray powder diffraction pattern having at least the following maxima: about 5.1°, 7.7°, 10.6°, 14.1°, 14.6°, 14.9° and 15.5° (2θ degrees).

6. The substantially pure crystalline form of claim 4, wherein the substantially pure crystalline form is characterized by the x-ray powder diffraction pattern of FIG. 2.

7. A method of producing a substantially pure crystalline form of 5-cyclopropyl-2-(4-fluorophenyl)-6-[(2-hydroxyethyl)(methylsulfonyl)amino]-N-methyl-1-benzofuran-3-carboxamide comprising recrystallizing crude 5-cyclopropyl-2-(4-fluorophenyl)-6-[(2-hydroxyethyl)(methylsulfonyl)amino]-N-methyl-1-benzofuran-3-carboxamide using at least one solvent and 5-cyclopropyl-2-(4-fluorophenyl)-6-[(2-hydroxyethyl)(methylsulfonyl)amino]-N-methyl-1-benzofuran-3-carboxamide seed of said crystalline form.

8. A method of producing a substantially pure crystalline form of 5-cyclopropyl-2-(4-fluorophenyl)-6-[(2-hydroxyethyl)(methylsulfonyl)amino]-N-methyl-1-benzofuran-3-carboxamide comprising the steps of:
(a) providing a solution of 5-cyclopropyl-2-(4-fluorophenyl)-6-[(2-hydroxyethyl)(methylsulfonyl)amino]-N-methyl-1-benzofuran-3-carboxamide in at least one solvent; and
(b) seeding the solution with 5-cyclopropyl-2-(4-fluorophenyl)-6-[(2-hydroxyethyl)(methylsulfonyl)amino]-N-methyl-1-benzofuran-3-carboxamide of said crystalline form.

9. The method of claim 8, wherein the substantially pure crystalline form A of 5-cyclopropyl-2-(4-fluorophenyl)-6-[(2-hydroxyethyl)(methylsulfonyl)amino]-N-methyl-1-benzofuran-3-carboxamide is produced.

10. The method of claim 8, wherein the substantially pure crystalline form B of 5-cyclopropyl-2-(4-fluorophenyl)-6-[(2-hydroxyethyl)(methylsulfonyl)amino]-N-methyl-1-benzofuran-3-carboxamide is produced.

11. The method of claim 8, wherein the solution of step (a) is provided by utilizing 5-cyclopropyl-2-(4-fluorophenyl)-6-[(2-hydroxyethyl)(methylsulfonyl)amino]-N-methyl-1-benzofuran-3-carboxamide in situ from a reaction mixture.

12. The method of claim 8, wherein the solution of step (a) is provided by dissolving 5-cyclopropyl-2-(4-fluorophenyl)-6-[(2-hydroxyethyl)(methylsulfonyl)amino]-N-methyl-1-benzofuran-3-carboxamide of any crystalline form in at least one solvent.

13. The method of claim 8, wherein step (a) is conducted at an elevated temperature.

14. The method of claim 13, wherein the elevated temperature is about 5° C. below the boiling point of the at least one solvent.

15. The method of claim 8, wherein the at least one solvent is selected from the group consisting of isopropyl alcohol, ethanol, ethyl acetate, acetonitrile, acetone, tetrahydrofuran, toluene, water, and combinations thereof.

16. The method of claim 8, wherein the at least one solvent is employed in an amount ranging from about 3 to about 20 times by weight of the amount of 5-cyclopropyl-2-(4-fluorophenyl)-6-[(2-hydroxyethyl)(methylsulfonyl)amino]-N-methyl-1-benzofuran-3-carboxamide.

17. The method of claim 8, wherein the solution is seeded with form A 5-cyclopropyl-2-(4-fluorophenyl)-6-[(2-hydroxyethyl)(methylsulfonyl)amino]-N-methyl-1-benzofuran-3-carboxamide seed crystals.

18. The method of claim 8, wherein the solution is seeded with form B 5-cyclopropyl-2-(4-fluorophenyl)-6-[(2-hydroxyethyl)(methylsulfonyl)amino]-N-methyl-1-benzofuran-3-carboxamide seed crystals.

19. The method of claim 8 further comprising the step of (c) cooling the solution.

20. The method of claim 19, wherein step (c) is conducted after step (b).

21. The method of claim 19, wherein step (c) is conducted prior to step (b).

22. The method of claim 19, wherein step (c) is conducted simultaneously with step (b).

23. The method of claim 8, wherein an amount of seed ranging from about 0.01% to about 2% by weight of the 5-cyclopropyl-2-(4-fluorophenyl)-6-[(2-hydroxyethyl)(methylsulfonyl)amino]-N-methyl-1-benzofuran-3-carboxamide in solution.

24. The method of claim 8, wherein seeding step (b) is carried out in portions.

25. The method of claim 19, wherein step (c) is carried out in steps.

26. The method of claim 19, wherein step (c) is carried out continuously.

27. The method of claim 19, wherein the solution is cooled to about 0° C.

28. The method of claim 8, wherein the solution is agitated.

29. The method of claim 8 further comprising the step of (d) adding an anti-solvent to the solution.

30. The method of claim 29, wherein the anti-solvent is heptane.

31. The method of claim 29, wherein step (d) is conducted after step (b).

32. The method of claim 29, wherein step (d) is conducted prior to step (b).

33. The method of claim 29, wherein step (d) is conducted simultaneously with step (b).

34. A substantially pure crystalline form of 5-cyclopropyl-2-(4-fluorophenyl)-6-[(2-hydroxyethyl)(methylsulfonyl)amino]-N-methyl-1-benzofuran-3-carboxamide produced according to the method of claim 8.

35. A pharmaceutical composition comprising:
(a) a therapeutically effective amount of a substantially pure crystalline form of 5-cyclopropyl-2-(4-fluorophenyl)-6-[(2-hydroxyethyl)(methylsulfonyl)amino]-N-methyl-1-benzofuran-3-carboxamide; and
(b) at least one pharmaceutically acceptable carrier, diluent, vehicle or excipient.

36. The pharmaceutical composition of claim 35, wherein the substantially pure crystalline form of 5-cyclopropyl-2-(4-fluorophenyl)-6-[(2-hydroxyethyl)(methylsulfonyl)amino]-N-methyl-1-benzofuran-3-carboxamide is form A or form B.

37. The pharmaceutical composition of claim 35, wherein the substantially pure crystalline form of 5-cyclopropyl-2-(4-fluorophenyl)-6-[(2-hydroxyethyl)(methylsulfonyl)amino]-N-methyl-1-benzofuran-3-carboxamide is characterized by the x-ray powder diffraction pattern of FIG. 1 or FIG. 2.

38. A method of treating hepatitis C comprising the step of administering to a subject in need of such treatment a therapeutically effective amount of a substantially pure crystalline form of 5-cyclopropyl-2-(4-fluorophenyl)-6-[(2-hydroxyethyl)(methylsulfonyl)amino]-N-methyl-1-benzofuran-3-carboxamide.

39. The method of claim 38, wherein the substantially pure crystalline form of 5-cyclopropyl-2-(4-fluorophenyl)-6-[(2-hydroxyethyl)(methylsulfonyl)amino]-N-methyl-1-benzofuran-3-carboxamide is form A or form B.

40. The method of claim 38, wherein the substantially pure crystalline form of 5-cyclopropyl-2-(4-fluorophenyl)-6-[(2-hydroxyethyl)(methylsulfonyl)amino]-N-methyl-1-benzofuran-3-carboxamide is characterized by the x-ray powder diffraction pattern of FIG. 1 or FIG. 2.
